(19) United States
(12) Patent Application Publication
(10) Pub. No.: US 2023/0136523 A1
(43) Pub. Date: May 4, 2023

(54) DEVICE FOR CONVERTING AMMONIA TO NITRIC OXIDE

(71) Applicant: Bedfont Scientific Limited, Harrietsham (GB)

(72) Inventors: Scott DEAMER-SMITH, Harrietsham (GB); Trevor SMITH, Harrietsham (GB); Waseem AHMED, Harrietsham (GB)

(21) Appl. No.: 17/766,970

(22) PCT Filed: Oct. 7, 2020
(86) PCT No.: PCT/GB2020/052477

§ 371 Date: Apr. 6, 2022

(30) Foreign Application Priority Data
Oct. 8, 2019 (GB) 1914549.9

(51) Int. Cl. A61B 5/08; C01B 21/28; C01B 21/26; B01J 23/86; A61B 5/087; A61B 5/097; G01N 33/00

(52) U.S. Cl. CPC A61B 5/082; C01B 21/28; C01B 21/265; B01J 23/862; A61B 5/087; A61B 5/097; G01N 33/0013

(57) ABSTRACT

In examples, there is a device for converting ammonia (NH₃) in a human breath sample to nitric oxide (NO). The device comprises a tube and a heater. The tube comprises an inlet, an outlet, and a wall defining an internal surface of the tube and an external surface of the tube. The wall comprises substantially the same material along a thickness from the internal surface of the tube to the external surface of the tube. The material is catalytic for conversion of ammonia to nitric oxide. The heater is configured to heat the wall.

### BACKGROUND

Ammonia given off in exhaled air can be used as an indicator of gastrointestinal diseases, in particular Helicobacter pylori (H. pylori) infection. H. pylori is one of the most common bacterial pathogens in humans and is now recognised as a worldwide problem. It causes chronic gastritis, peptic ulcer disease, and lymphoproliferative disorders and is a major risk factor for gastric cancer.

At the present time there are a number of different tests that are available to test for the presence of the H. pylori infection, although they do not provide instant results and can be invasive (e.g. an endoscopy). These tests are also expensive to perform and involve sending samples to a laboratory to be tested which is time consuming and expensive. There is therefore a need for devices which test for the presence of the H. pylori infection and give a reading quickly.

Some devices in the art for analysing breath samples measure the concentration of ammonia in the breath sample directly by using an ammonia detector. However, such measurements can be inaccurate and it is desirable to provide a device with more accuracy.

### BRIEF DESCRIPTION OF THE DRAWINGS

FIGS. 1A and 1B are schematic diagrams of a device in accordance with examples.

FIGS. 2A and 2B are block diagrams of a device in accordance with examples.

FIG. 3 is a flow diagram of a method in accordance with examples.

FIG. 4 is a flow diagram of another method in accordance with examples.

### DETAILED DESCRIPTION

In devising examples described herein, the inventors have found that accurately detecting the amount of ammonia in a breath sample presents certain challenges. For example, ammonia present in a breath sample has a propensity to adhere to surfaces which the ammonia contacts. This adhesion may limit the amount of ammonia which reaches an ammonia detector, thereby reducing the accuracy of any ammonia concentration reading. Therefore, according to examples described here, there is provided a device which allows for more accurate measurement of ammonia in a breath sample, for example by effectively reducing the surface area which the ammonia will contact before reaching a detector.

According to examples there is provided a device for converting ammonia (NH₃) in a human breath sample to nitric oxide (NO). A human breath sample may also be referred to as “exhaled air”.

The device comprises a tube for receiving a human breath sample, and a heater for heating the wall of the tube (e.g. to heat a human breath sample received in the tube). The tube comprises an inlet. The human breath sample passes through the inlet to reach the inside of the tube. The tube also comprises an outlet. The human breath sample passes through the outlet as it leaves the tube. The tube further comprises a wall which defines an internal surface of the tube and an external surface. Thus, an inner surface of the wall e.g. corresponds with, and thus provides, the internal surface of the tube. In examples, when the tube receives a human breath sample, the human breath sample is in contact with the internal surface of the tube. The shortest distance between the internal surface and the external surface of the tube may be referred to as the wall thickness. The volume between the inlet and the outlet of the tube and contained by the internal surface of the tube may be referred to as the inside, cavity or lumen of the tube, or as the oxidation chamber.

In examples, the heater may be configured to heat the tube, and thus the human breath sample in the tube, to a temperature of from 500 to 1000° C. (degrees Celsius), from 600 to 900° C., or from about 700° C. to about 800° C., in the presence of oxygen. In some examples, the heater is configured to heat the tube to a temperature greater than or equal to 640° C. The oxygen may originate from the exhaled air, in which case the oxygen is at a level of greater than 15 wt % of the exhaled air, such as from 15 to 17 wt %.

The device is configured to convert ammonia to nitric oxide in the tube. Nitric oxide has a lower propensity for adhering to surfaces than ammonia. Accordingly, converting the ammonia in the breath sample to nitric oxide provides an analyte which may more reliably reach a detector, and thereby give a more accurate indication of the concentration of analyte in the breath sample. In examples, the ammonia is converted to nitric oxide as close to the patient as possible, to reduce the distance the ammonia travels in the test apparatus (and thereby reduce the surface area which the ammonia may contact).

Ammonia present in the exhaled air undergoes thermal oxidation upon heating, as follows:

4NH₃+5O₂→4NO+6H₂O

Performing the thermal oxidation in the presence of a catalyst assists in providing a high efficiency of conversion of ammonia to nitric oxide in the device, e.g. 90% or higher, such as substantially all of the ammonia present in the breath sample. Surprisingly, the inventors have discovered that forming the tube in which the breath sample is heated itself of catalytic material (for example, material which is catalytic for the oxidation of ammonia to nitric oxide) gives notably better catalytic performance. The wall of the tube comprises substantially the same material along its thickness. The material at the internal surface of the tube will have substantially the same composition as the material at the external surface of the tube, as well as all the material therebetween. In this context, “substantially” means for example that the wall comprises small amounts of material which is not the catalytic material, such as impurities derived from the manufacturing process, or build-up/fouling at a surface of the tube deriving from use of the device, but which has a negligible impact on the catalytic function of the tube. In examples, a tube having substantially the same composition along its thickness will have a catalytic activity which deviates from a corresponding tube comprising only the catalytic material along its thickness by less than 5%, 2%, 1%, or 0.1%. In some examples, the tube comprises only the catalytic material along its thickness. In at least some examples described herein the only material catalytic for the oxidation of ammonia to nitric oxide is the catalytic material of the wall of the tube. Thus, in examples, the lumen of the tube is substantially free from any catalytic material.

As compared with a tube having an inner coating of catalytic material, the tube of the device in examples described herein having a substantially constant catalytic composition, and without an inner coating of catalytic material, means that the tube can undergo more heating-cooling cycles without substantial degradation. For example, where a tube includes a wall and an inner coating of a different, catalytic material along the internal surface of the tube, the wall and inner coating may respond differently during heating cycles. In contrast, the absence of an inner coating on the tube as described herein avoids the risk of such degradation.

Further, manufacture of the device described herein may be cheaper and/or simpler than the manufacture of a device comprising a laminate heating tube (e.g. with an inner coating of catalytic material). To provide an inner coating to a heating tube expensive and time-consuming process must be carried out, such as electrostatic powder coating. In contrast, the present inventors have discovered that such an expensive and time-consuming manufacturing process may be obviated by forming the heating tube from a catalytic material such that the tube has a substantially constant composition along its width.

In some examples, the wall comprises a metal, such as a metal alloy. In some examples, the wall comprises stainless steel. In some examples, the wall comprises stainless steel in an amount of at least 95 wt %, 96 wt %, 97 wt %, 98 wt % or 99 wt % of the wall. Surprisingly, the inventors have identified that, when heated to a suitable temperature, stainless steel is catalytic in the thermal oxidation of ammonia to nitric oxide. In particular examples, the steel is 316 stainless steel (steel containing 16% chromium, 10% nickel and 2% molybdenum). The inventors have identified that the wall comprising 316 stainless steel also has properties beyond its catalytic behaviour, such as higher resistance to corrosion and oxidation. This higher resistance to corrosion and oxidation may in turn provide an increased working lifetime to the device. Without wishing to be bound by theory, it is believed that the high molybdenum content in particular contributes to the observed corrosion resistance.

In some examples, the device is configured such that it can be held by hand in use. The inventors have identified that the device may efficiently convert ammonia to nitric oxide and be configured to be hand-held by configuring the tube such that the ratio between the distance within the tube from the inlet to the outlet, and the shortest distance from the inlet to the outlet, is greater than or equal to 2:1. In some examples, the ratio is greater than or equal to 2:1, 5:1, or 10:1.

The ‘distance within the tube’ may be referred to as the shortest available flow path for the breath sample to pass from the inlet of the tube, along the inside of the tube, to the outlet of the tube. The distance within the tube refers only to the flow path between the inlet and outlet of the tube which is formed of substantially the same material along its wall thickness. The distance within the tube does not include the flow path of the breath sample through any other part of the device, such as through a mouthpiece which connects to the inlet of the tube, or a further conduit connecting the outlet of the tube to a sensor.

The distance within the tube should be long enough that, in use, at least 90% or substantially all (e.g. 95% or greater) of the ammonia which passes through the tube is converted to nitric oxide before leaving the outlet. In some examples, the distance within the tube is greater than or equal to 0.5 metres (m), 1 m, 1.5 m, 2 m, or 2.5 m. In some examples, the distance within the tube is from 0.5 to 3 m, from 1 to 2.5 m, or from 1.5 m to 2.2 m.

The shortest distance between the inlet and the outlet of the tube is for example be a distance which can be accommodated in a handheld device. In some examples, the shortest distance between the inlet and the outlet of the tube is less than or equal to 30 cm, 20 cm, 16 cm, or 9 cm.

In some examples the tube has an outer diameter (OD) of from 1 to 10 millimetres (mm), or from 2 to 7 mm, or from 1 to 5 mm, or from 2 to 3 mm.

In some examples the tube has an internal diameter (ID) of from 0.5 to 8 mm, or from 0.5 to 5 mm, or from 1 to 3 mm, or from 1.5 to 2 mm.

A tube having a distance within the tube of 1.95 m, a shortest distance from inlet to outlet of 9 centimetres (cm), an outer diameter of 2.38 mm and an inner diameter of 1.78 mm has been found to be suitable.

A tube having a distance within the tube of 1.95 m, a shortest distance from inlet to outlet of 15 centimetres (cm), and an internal diameter of 6 mm has also been found to be suitable.

The tube may have any suitable shape. In some examples, the tube has a substantially helical shape. In these examples, the heater may be provided as a core around which the tube is wound. In some examples, the outer diameter of the helix (the largest lateral dimension of the helix) is from 5 to 40 mm, or from 10 to 30 mm, or from 15 to 25 mm.

As stated above, the device is adapted to allow thermal oxidation of ammonia to a sufficient degree to allow analysis of the resultant NO (nitric oxide) levels. 100% oxidation of ammonia is not necessarily required, but oxidation of 90% or greater is generally desirable. In examples, the device is adapted to provide a consistent degree of ammonia oxidation, within acceptable tolerances. For example, the device may be adapted, for example with appropriate dimensions of the tube, so that for different samples (possibly of different volumes and/or a different proportion of ammonia), the proportion of ammonia (input via the inlet) relative to the proportion of NO (output via the outlet) converted in the tube from the input proportion of ammonia, is sufficiently consistent. For example, this could be expressed as a conversion ratio of output NO:input NH₃, which for a range of different samples (possibly of different volumes, from different patients, and/or with different input ammonia proportions) is approximately the same, for example with a deviation of less than 5%, less than 3%, or less than 1%. So, in some examples, the device is adapted such that the proportion of ammonia oxidised to NO in each breath sample is 90±1% of the ammonia present in the breath sample.

In examples the heater has any shape suitable for supplying heat to the tube. In some examples the heater is elongate, having a length which is greater than its width and its height (such as at least 2, 3 or 4 times greater). In these examples, the heater may be cylindrical. The tube and heater may be arranged such that the tube forms a coil around the cylindrical heater. In particular examples the heater is a cartridge heater, and the tube forms a coil around the length of the cartridge heater. In other examples, the heater is tubular and surrounds the tube along its length.

The temperature of the heater (and in turn the temperature of the wall of the tube) may be controlled by a suitable thermostat, e.g. temperature control circuit with a thermocouple, which serves to monitor the temperature. The temperature may be regulated by means of a thermal feedback loop—data from the thermocouple is supplied to a processor, which will compare the received data with a predetermined value, and instruct power to be supplied to the heater depending on the data depending on the comparison between the data and the predetermined value. This process may be repeated at any suitable interval.

In some examples, the temperature of the heater is displayed at a user interface. The heater may comprise a high temperature cut-out device as a safety feature to reduce the risk of damage to other components of the device or to the patient/user.

The device may have insulation to protect the user and the other components of the device from exposure to excessive heat. This may be particularly important where the device is configured to be a handheld device. In some examples, the tube and heater are together wrapped with a suitable thermal insulator such as glass wool or thermal cement. The thermal insulator-covered tube-and-heater assembly may be further encased in plastics to provide further thermal insulation between a user and the components of the device which reach high temperatures.

In examples the heater is configured to receive alternating-current electric power from a power source to thereby provide heat to the wall. Suitably, the heater may be configured to receive power from a mains supply. The heater may be configured to receive power from a dock, power point, charging station, cradle, and the like (such terms may be used interchangeably later). The charging station may in turn receive power from a mains power supply. Power received from a mains power supply (otherwise known as domestic, grid or wall power) will be dependent on where the device is used, but generally a mains supply supplies power having a voltage of at least 100 volts (V) and a frequency of at least 40 Hertz (Hz).

In some examples, the heater is removably electrically connected to the power source. In examples, where the device is configured to receive power from a charging station, the charging station and device are configured such that the device and charging station are repeatedly connectable and disconnectable. In some examples, the charging station is configured to support the device while the device and charging station are connected. For example, the charging station has a recess in which the heater may be deposited. The charging station and the device may have corresponding connectors through which electricity flows. For example, the charging station has one or more female electrical connectors, and the device has one or more male electrical connectors. The charging station may be configured to supply power to the heater which is of a similar voltage and frequency as mains power. In some examples, the charging station is configured to supply electricity having a voltage of at least 100 V and a frequency of at least 40 Hz.

As mentioned hereinabove, providing the device in a handheld form factor presents particular problems, such as ensuring the safety of the patient or practitioner during use. Accordingly, in some examples, the heater is disconnected from a mains power supply while a breath sample is taken and the ammonia in the breath sample converted to ammonia. In some examples, the heater is disconnected from a power supply which supplies electricity having a voltage of at least 100 V and a frequency of at least 40 Hz. In some examples, the material forming the tube is selected to have thermal properties such that the tube may be rapidly heated (to avoid long warmup times before carrying out a test), but retain heat for a long enough period for the tube to receive a breath sample and convert substantially all of the ammonia in the sample to nitric oxide without the heater receiving electrical power.

In some examples the heater is configured to be repeatedly during use connected to the power source until the heater has heated the wall to a first temperature greater than a second temperature at which oxidation of ammonia to nitric oxide occurs, before an exhaled air sample is received within the tube, and disconnected from the power source before the human breath sample is received in the tube. In some examples, the first temperature is 600° C., 650° C., 700° C., or 750° C. In some examples, the second temperature is 550° C., 600° C., or 640° C. In some examples, the device is configured such that, after disconnection of the device from the power source, the wall has a temperature greater than or equal to the second temperature for at least thirty seconds, one minute or two minutes from disconnection.

For example, the device is configured such that, if the heater is supplied with electric power such that the heater reaches a temperature of at least 700° C. (e.g. a temperature of from 700° C. to 800° C.) and is subsequently disconnected from the power source, the tube has a temperature of at least 640° C. for at least thirty seconds after disconnection from the power source, or at least one minute, or at least two minutes seconds.

The temperature of the wall of the tube may be measured by a temperature sensor (e.g. a thermocouple) at a surface of the tube. For example, the temperature of the wall is measured by a temperature sensor in contact with any portion of the internal surface of the tube or the external surface of the tube. In examples, the thermal conductivity of the tube and the arrangement of the tube together with the heater means that there is little variation in temperature along the tube, and thus a measurement of the temperature of the wall of the tube can be suitably taken at any part of a surface of the tube.

In general, the device should be configured such that at least 90%, or substantially all of the ammonia in the human breath sample is oxidised to nitric oxide during passage between the inlet and the outlet of the tube. This may be affected by the tube dimensions, a catalytic activity of the material, or heat energy outputtable by the heater such that substantially all ammonia in the human breath sample is oxidised to nitric oxide during passage between the inlet and the outlet.

This configuration may include selecting a material for the tube having a suitable heat capacity. The device according to any preceding claim, wherein the material of the tube wall has a specific heat capacity (cₚ) of from 400 to 600 Joules per kilogrammes Kelvin (J/(kg·K)), or from 450 to 550 J/(kg·K).

This configuration any alternatively or additionally involve selecting a tube having a suitable mass. Taken together, the specific heat capacity and the mass of the tube will define the heat capacity of the tube. The heat capacity in combination with the tube wall thickness will determine the tube's thermal behaviour after the heater stops receiving power from a power source. In some examples, the tube has a mass of from 10 to 50 g (grammes), or from 20 to 40 g, or from 25 to 35 g. In some examples, the tube has a wall thickness of from 0.1 to 1 mm, or from 0.1 to 0.5 mm, or from 0.15 to 0.35 mm.

In examples the device comprises a nitric oxide (NO) sensor for detecting the level of nitric oxide within the heated exhaled air output from the outlet of the tube, in order to measure the concentration of ammonia in the original exhaled air sample. The amount of NO in the sample will correspond to the amount of ammonia originally present. A suitable NO sensor are available from the vendor Membrapor, Birkenweg 2 8304 Wallisellen, Switzerland.

The portion of the device containing the tube and heater may conveniently be referred to herein as the “heating portion”. Similarly, the portion of the device containing the nitric oxide sensor may conveniently be referred to as the “sensing portion”.

The sensing portion is in fluid communication with the heating portion—in particular, the outlet of the tube is in fluid communication with the NO sensor, allowing the heated human breath sample to pass from the tube to the NO sensor. The outlet and the NO sensor may be connected by any suitable means, such as a conduit formed of plastics.

The sensor portion may further comprise a pump configured to extract a portion of the oxidised exhaled air sample for detection by the sensor. In this example, the extracted portion is drawn through a solenoid valve to be brought into contact with the sensor, into which it diffuses.

The sensor may be capable of sensitive measurement of concentrations of NO from parts per billion (ppb) levels to parts per million levels (ppm). In some examples, the sensor is capable of detecting levels of NO at around 0 to 300 ppb. In some examples, the minimum detection limit is 5 ppb.

Typically, the sensor is an electrochemical gas sensor, as it is temperature stable. This negates the need for heating or cooling of the sensor or heated exhaled air during use. Furthermore, it offers space saving opportunities in the apparatus design and simplicity of design and construction of the apparatus.

The electrochemical sensor may comprise a filter capable of removing acid gases, such as those formed as a by-product of thermal oxidation, where the ammonia reacts with too much oxygen, and any particulate objects remaining in the sample. The filter may also be capable of acting as a further moisture filter.

The electrochemical sensor may have 5% resolution, in that 5% is the smallest change it can detect in the quantity of NO, and may provide an output which is linearly proportional to the NO concentration. The production of a linear output makes the use of an electrochemical sensor advantageous over prior art sensors as it negates the need to linearise the output before the measurement can be determined. Furthermore, such features of the electrochemical sensor may enable the device described herein to be portable and to provide real time ammonia measurements. In some examples, the electrochemical sensor has a resolution of 1 ppb.

The electrochemical device may have cross sensitivity to nitrogen (100% N₂ being detected up to a level of −0.05 ppb), carbon dioxide (1.12% CO₂ being detected up to a level of 17.3 ppb) and carbon monoxide (45 ppm CO being detected up to a level of 17.6 ppb). In some examples, the device, e.g. the electrochemical sensor, has an inbuilt CO filter to remove any CO present in the heated exhaled air sample.

Typically, the electrochemical sensor has a 350 mV (milli Volts) bias and an operating pressure of 1 atm+/−10%. The sensor may be suitable for continuous use at a temperature between 10° C. and 30° C. and at a relative humidity of 25% to 75%, and intermittent use at a temperature between 0° C. and 35° C. and at a relative humidity of 0% to 100%.

The heating portion and/or sensor portion may each contain an independent power source, such as a battery, such that it is portable and can be used when disconnected from the power grid. The sensor portion may comprise a biasing pin to maintain bias. In some examples, the sensor portion comprises a 350 mV biasing pin. The sensor portion may contain a back-up battery to ensure power to a memory within the sensor portion which contains software and/or to the gas sensor. In some examples, the heating portion does not contain an independent power source and is configured to receive power from an external power source, such as a mains power source of a charging station.

In examples the heating portion is detachable or disconnectable from the sensing portion. In these examples, the heating portion and the sensing portion each comprise corresponding interface means to allow the portions to be connected together. Advantageously, the connection between the heating portion and the sensing portion is substantially airtight. In one example, the connection comprises a plug and socket arrangement.

The device may further comprise a patient contact portion. The patient supplies a breath sample to the tube of the heating portion via the patient contact portion, e.g. the patient may exhale into the patient contact portion. The patient contact portion is for example a mouthpiece, a facemask, a nasal breath sampling means or a combination of one or more of these. A mouthpiece is simple to use. Further, in use, a mouthpiece can extend towards the back of the user's mouth, allowing capture of a breath sample containing only ammonia without allowing the ammonia to adhere to the user's oral cavity and avoid detection by the device. Moreover, this arrangement only captures ammonia from the airways, and avoids contamination from ammonia produced by mouth flora, plaque or saliva. In some examples, in use the device and/or mouthpiece provide back pressure while the user exhales into the device. This back pressure closes the velum (soft palate), thereby preventing or reducing contamination of the sample with ammonia from the nasal cavity. In some examples, the back pressure is from 8 to 15 millibar.

As between the heating portion and the sensing portion, the patient contact portion may be detachable from the heating portion. In these examples, the patient contact portion and the heating portion. Each comprise corresponding interface means to allow the portions to be connected together in a substantially airtight manner (e.g. so that no, or negligible (e.g. 5% or less) air leaks out where the patient contact portion and the heating portion interface). In some examples, at least part of the patient contact portion and/or interface means is heated to a temperature above ambient temperature during use, to reduce adsorption of ammonia before it reaches the heating portion.

As discussed hereinabove, it is envisaged that the device will be shaped and sized such that it is suitable for portable operation. This allows the unit to be conveniently used in a variety of settings, either with or without a trip to a trained clinician being necessary. This makes the apparatus particularly useful for H. pylori testing as triage prior to an endoscopy procedure, as it does not require the presence of bulky table-top equipment. Additionally, smaller apparatus for testing the concentration of ammonia provides general space saving benefits.

The device may obviate the need to have an additional gas storage chamber and a pumping device to ensure a controlled flow rate to the sensor. This further facilitates a small and simple testing apparatus and reduces the cost of such apparatus, making it attractive in all primary care settings and developing countries where H. pylori is more prevalent.

The device of examples is used for measuring levels of gaseous ammonia in an oral breath sample, but may additionally or alternatively be used for a nasal breath sample, or other gas forms from a subject.

According to further examples, there is a method of manufacturing a device for converting ammonia (NH₃) in a human breath sample to nitric oxide (NO). In the method a material catalytic is selected for conversion of ammonia to nitric oxide. Then a tube is formed from the material, the tube having an inlet, an outlet, and a wall defining an internal surface of the tube and an external surface of the tube, the wall comprising the material along a thickness from the internal surface of the tube to the external surface of the tube. The tube and the heater are arranged such that in use the heater heats the tube in a manner required for conversion of ammonia to NO in an exhaled breath sample.

The selection of the material may be based on any of the parameters described hereinabove.

The tube may be prepared according to any suitable process, such as welding (to produce a welded tube) or methods of forming seamless tubes (such as extrusion). In some examples, the tube is prepared by drawing, such as cold-drawing.

The method may further comprise arranging thermal insulation around the tube and heater. This may comprise arranging glass fibre or thermal cement around the tube and heater. Suitably, little or no thermal insulation is arranged between the tube and heater so that the efficiency of heating the tube by the heater is not adversely affected. In some examples, where the heater is elongate and the tube is arranged as a coil around the heater such that a surface of the tube directly abuts the heater, arranging thermal insulation around the tube and heater may comprise covering at least 50, 60, 70, 80 or 90% of the surface of the tube and heater exposed to the atmosphere with thermal insulation.

The method may further include encasing the tube, heater, thermal insulation and, in some examples, further components, in a housing. The housing may also be thermally insulating. In some examples, the housing comprises plastics. In some examples, such as those wherein the housing comprises plastics, the thermal insulation and the housing are arranged such that, in use, the external surface of the housing which may be handled by a user reaches a temperature of no more than 45° C. In some examples, the housing comprises aluminium. In some examples, such as those wherein the housing comprises aluminium, the thermal insulation and the housing are arranged such that, in use, the external surface of the housing which may be handled by a user reaches a temperature of no more than 41° C.

According to further examples, there is a method of measuring the concentration of ammonia in a human breath sample with a device for converting NH₃ in a human breath sample to nitric oxide (NO). The device may be any device described herein.

The method comprises heating the wall of the tube to a temperature equal to or greater than 600° C., or equal to or greater than 700° C. In some examples, the method comprises heating the wall of the tube to a temperature of from 600° C. to 900° C., or from 700 to 800° C. Heating the wall of the tube may comprise supplying power to the heater from a power supply, such as an alternating-current electric power source as described hereinabove, and thus supplying thermal energy to the wall of the tube from the heater. In some examples, heating the wall of the tube comprises supplying power from a direct-current electric power source. The temperature to which the wall of the tube is heated while power is supplied from the power supply may be referred to as “the first temperature”.

The method further comprises supplying the human breath sample to the inlet of the tube such that the human breath sample passes along the tube and through the outlet to the nitric oxide sensor, wherein at least 90% of the ammonia present in the human breath sample is converted to nitric oxide before contacting the nitric oxide sensor. Supplying the human breath sample may comprise a patient breathing into a patient contact means which is connected to the tube. The device may include further pumps or regulators to control the flow of breath sample through the tube.

In some examples, where the wall of the tube is heated by supplying power to the heater from a power supply, the power supply is disconnected from the heater before supplying the human breath sample to the tube. As explained hereinabove, this may beneficially affect the safety of the patient and/or clinician during use. In some examples, the human breath sample ceases to be supplied to the inlet of the tube before the temperature of the tube wall drops to a second temperature lower than the first temperature, such as 600° C., or 640° C., or 700° C.

The method further includes detecting the amount of nitric oxide present in the heated human breath sample with the nitric oxide sensor to provide a measurement of the concentration of ammonia in the human breath sample.

The method may include providing to the user an indication of the rate of exhalation so that they can alter the exhalation rate such that it falls within a desired range. This may conveniently be achieved using a floating ball or similar arrangement electronic interface.

Once the sample has passed through the tube and out of the outlet, the method may comprise extracting a portion of the heated sample air for analysis within the sensor. This may involve the use of a pump, e.g. contained in the sensor portion, to effect extraction.

Suitably the rate of exhalation is for example as defined above for a time sufficient for sampling to occur.

In some examples, the method further comprises drawing a nitric oxide free sample of air and/or ambient air into the sensor device to calibrate the nitric oxide sensor.

The method may comprise comparing the result of the method with an expected value. From this a diagnostic or prognostic indication may be derived, e.g. the diagnosis of H. pylori infection.

According to one example, there is provided the use of a tube comprising an inlet, an outlet, and a wall defining an internal surface of the tube and an external surface of the tube, the wall comprising substantially the same material along a thickness from the internal surface of the tube to the external surface of the tube, the material comprising stainless steel, in catalysing the conversion of ammonia to nitric oxide in a human breath sample.

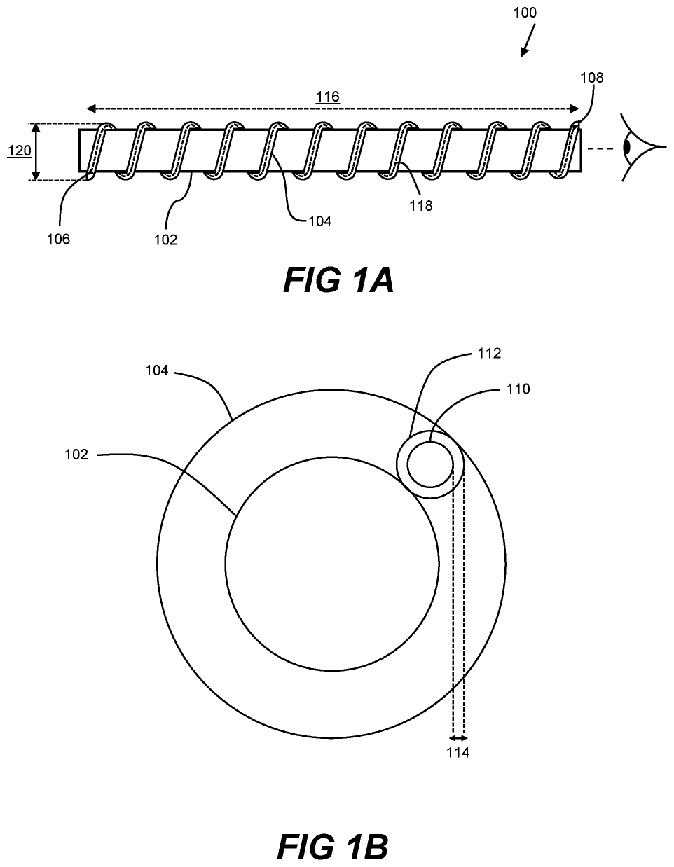

With regard to FIGS. 1A and 1B, there is shown a schematic of a heating portion of the device described herein. The heating portion 100 comprises a heater 102 and a tube 104 in accordance with examples described previously. Thus, the tube 104 has an inlet 106 and an outlet 108, an internal surface 110 and an external surface 112. The tube has a wall thickness 114.

FIG. 1A is a side elevation of the heating portion; FIG. 1B is a front elevation of the heating portion, viewed along the sight line indicated in FIG. 1A.

In use a breath sample from a patient enters the heating portion 100 via patient interface means (not shown) and then passes into the tube 104 for example via interface means (not shown) and the inlet. In the tube 104, the breath sample is heated to at least 640° C. by the heater 104 in the presence of oxygen and in contact with the inner surface 110 to bring about conversion of ammonia to nitric oxide. The heater 102 is powered by a mains supply, which is removably connectable.

FIG. 1A also indicates some parameters of the tube 104 in dashed lines. Dimension 116 indicates the shortest distance 112 between the inlet 106 and the outlet 108 of the tube 104. Dimension 118 indicates the shortest distance within the tube 104 from the inlet 106 to the outlet 108. The shortest distance 118 within the tube 104 corresponds to the flow path of the breath sample through the tube 104 during use, and the longitudinal axis which extends along the tube 104. Tube 104 is provided as a helix, in other words the form of the tube has a helical shape. Dimension 120 indicates the outer diameter of the helix (the largest lateral dimension of the helix).

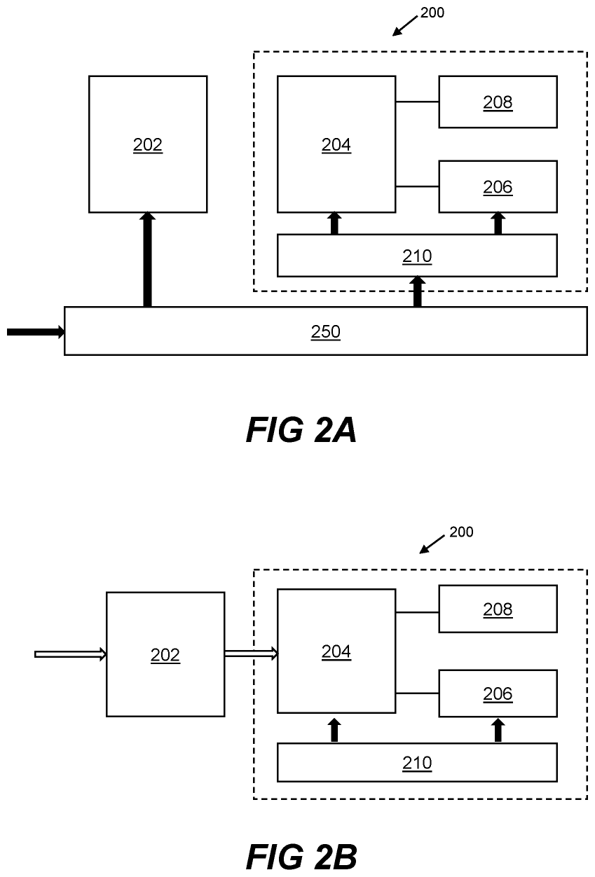

FIGS. 2A and 2B show a schematic of a device 200 comprising a heating portion 202 corresponding to the heating portion 100 described hereinabove, and further components. The device comprises a sensor portion 204, user interface 206, serial communications 208 and a battery 210.

FIG. 2A shows the device in connection with a charging station 250. The charging station 250 receives power from a mains power supply.

As shown in FIG. 2A, before a breath sample is supplied to the device 200, the device 200 is connected to the charging station 250 so that power is supplied from the charging station 250 to the heater in the heating portion 202 (indicated by shaded arrows). Power may also be supplied from the charging station to charge/recharge the battery 210.

The device 200 is connected to the charging station 250 until the heater reaches a first temperature, such as 700° C. (e.g. a temperature of from 700° C. to 800° C.). Once the heater reaches the first temperature and before a breath sample is supplied to the device 200, the device 200 is disconnected from the charging station 250.

As shown in FIG. 2B, a breath sample is supplied to the heating portion 202 (indicated by unshaded arrows). Once the ammonia present in the breath sample has been converted to nitric oxide in the heating portion 202, at least a portion of the heated sample passes from the heating portion 202 into the sensor portion 204. In one example, the portion of the heated sample is provided to the sensor portion 204 at a rate of 350 ml/min (millilitres per minute). As stated previously, it is not necessary that the heating device 102 necessarily has a 100% conversion rate but rather that at least 90% of the ammonia present in the breath sample is converted to nitric oxide. The use of a calibration routine for the sensor portion 204 (as discussed in greater detail below) may enable a conversion rate of less than 100% to lead to an accurate measurement of ammonia in the breath sample. The term “heated sample” as used herein refers in examples to a breath sample which has passed through the tube and has been heated such that ammonia has been converted to NO. The heated sample subsequently cools after exiting the tube. For the avoidance of doubt, the term “heated sample” does not mean that the sample has an elevated temperature; rather, it means that the sample has been heated such that ammonia has been converted to NO, but which covers a subsequently cooled sampled.

The sensor portion 204 comprises an electrochemical NO sensor which is mounted within the sensor portion 204 such that heated sample passing from the heating portion 202 into the sensor portion 204 passes over the relevant portion of the NO sensor before exiting the device through an exhaust port (not shown). Suitably the NO sensor is arranged such that a gas entry surface faces into a diffusion cavity through which the heated sample passes. The heated sample may have cooled prior to contacting the NO sensor. Such cooling may reduce thermal damaging of the sensor.

The amount of NO present in the heated sample is detected by the sensor portion 204, which communicates this information to user interface 206. Additionally, or alternatively, the information provided by the NO sensor is processed within the apparatus (for example by memory and at least one processor) such that an indication of the concentration of ammonia present in the breath sample is provided at user interface 206.

The user interface 206 comprises a display, and input means to allow a user of the device to operate the device. A touch sensitive LCD (liquid crystal display) is an example of a system which combines both input and display functions. The display, amongst other functions, for example indicates the amount of NO in the heated sample, as detected by the sensor portion 204, or the concentration of ammonia present in the original breath sample. However, in alternative examples, the display is in the form of LED indicators, or an LCD.

The sensor portion 204 communicates the amount of NO detected in the heated sample to serial communications 208, which is connected to any electronic storage device, for example as part of a computer. The computer compares the amount of NO detected with known values to ascertain whether the patient has a gastrointestinal disease, such as H. pylori infection.

The battery 210 supplies power to at least the sensor portion 204 and user interface 206 while the breath sample is supplied to the device 200 and the ammonia content measured. In some examples, the batter 210 does not supply power to the heating portion 202.

In some examples, when the device 200 is disconnected from the charging station 250, the heating portion 202 is electrically insulated from the sensor portion 204, user interface 206, serial communications 208 and battery 210 (e.g. the portion enclosed in dashed lines in FIGS. 2A and 2B). Electrically insulating the heating portion 202 from the other parts depicted may reduce the risk of injuring a patient or clinician while supplying a breath sample to the device 200 e.g. by reducing the chance of electric shock.

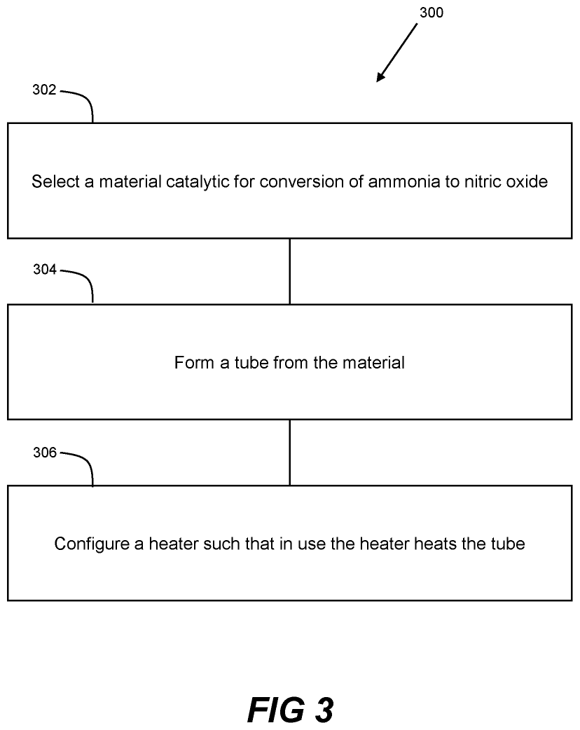

FIG. 3 is a flow diagram depicting a method 300 according to an example of the present disclosure. The method 300 is a method 300 of manufacturing a device for converting ammonia (NH₃) in a human breath sample to nitric oxide (NO). The device may suitably by any device described herein.

The method 300 comprises selecting a material catalytic for conversion of ammonia to nitric oxide 302. The method 300 further comprises forming a tube from the material 304. The tube has an inlet, an outlet, and a wall defining an internal surface of the tube and an external surface of the tube, the wall comprising the material along a thickness from the internal surface of the tube to the external surface of the tube. The method 300 further comprises configuring a heater such that, in use, the heater heats the tube 306.

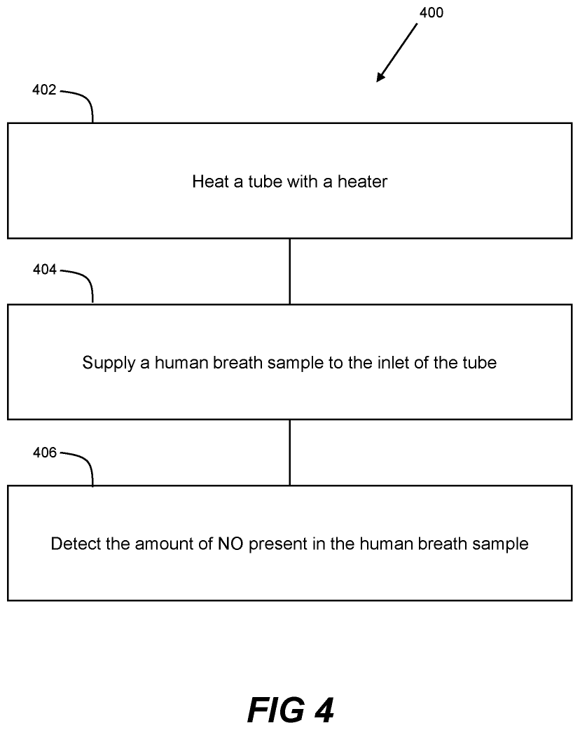

FIG. 4 is a flow diagram depicting a method 400 according to an example of the present disclosure. The method 400 is a method of measuring the concentration of ammonia (NH₃) in a human breath sample with a device for converting NH₃ in a human breath sample to nitric oxide (NO). The device may suitably by any device described herein.

The method 400 comprises heating a tube with a heater 402. The heater heats the tube to a temperature equal to or greater than 600° C. The tube comprises an inlet, an outlet, and a wall comprising an internal surface of the tube and an external surface of the tube, the wall comprising substantially the same material along a thickness from the internal surface of the tube to the external surface of the tube, the material catalytic for conversion of ammonia to nitric oxide.

The method 400 further comprises supplying the human breath sample to the inlet of the tube 404. The human breath sample is supplied such that the human breath sample passes along the tube and through the outlet to a nitric oxide sensor in fluid communication with the outlet, wherein the human breath sample is heated and at least 90% of the ammonia present in the human breath sample is converted to nitric oxide before contacting the nitric oxide sensor.

The method 400 further comprises detecting the amount of nitric oxide present in the heated human breath sample 406. The amount of nitric oxide is detected with the nitric oxide sensor. The measurement of nitric oxide concentration provides a measurement of the concentration of ammonia in the human breath sample.

The above examples are to be understood as illustrative. Further examples are envisaged. It is to be understood that any feature described in relation to any one example may be used alone, or in combination with other features described, and may also be used in combination with one or more features of any other of the example, or any combination of any other of the example. Furthermore, equivalents and modifications not described above may also be employed without departing from the scope of the accompanying claims.

1. A device for converting ammonia (NH₃) in a human breath sample to nitric oxide (NO), comprising:
a tube comprising:
an inlet;
an outlet; and
a wall defining an internal surface of the tube and an external surface of the tube, the wall comprising substantially the same material along a thickness from the internal surface of the tube to the external surface of the tube, and the material catalytic for conversion of ammonia to nitric oxide; and
a heater configured to heat the wall.

2. The device according to claim 1, wherein a ratio between:
the shortest distance within the tube from the inlet to the outlet; and
the shortest distance from the inlet to the outlet is greater than or equal to 2:1.

3. The device according to claim 1, wherein the material catalytic for conversion of ammonia to nitric oxide comprises stainless steel.

4. The device according to claim 3, wherein the stainless steel is 316 stainless steel.

5. The device of claim 1, wherein at least one of: dimensions of the tube, a catalytic activity of the material, or heat energy outputtable by the heater is such that substantially all ammonia in the human breath sample is oxidised to nitric oxide during passage between the inlet and the outlet.

6. The device of claim 1, the heater configured to be repeatedly during use i) connected to a power source until the heater has heated the wall to a first temperature greater than a second temperature at which oxidation of ammonia to nitric oxide occurs, before an human breath sample is received within the tube; and ii) disconnected from the power source before the human breath sample is received in the tube.

7. The device of claim 6, the device configured such that, after disconnection of the device from the power source, the wall has a temperature greater than or equal to the second temperature for at least thirty seconds.

8. The device of claim 6, wherein the second temperature is at least 640° C.

9. The device according to claim 1, wherein the tube has a substantially helical shape.

10. The device according to claim 4, wherein the heater is elongate and the tube is arranged around the heater.

11. The device according to claim 1, wherein the shortest distance within the tube from the inlet to the outlet is greater than or equal to 1 metre.

12. The device according to claim 1, wherein the shortest distance between the inlet and outlet is less than or equal to 30 centimetres.

13. The device according to claim 1, wherein the material of the tube wall has a specific heat capacity (cₚ) of from 400 to 600 J/(kg·K).

14. The device according to claim 1, wherein the tube has a mass of from 10 to 50 g.

15. The device according to claim 1, wherein the tube has a wall thickness of from 0.1 to 1 mm.

16. The device according to claim 1, comprising a nitric oxide sensor in fluid communication with the outlet.

17. A method of manufacturing a device for converting ammonia (NH₃) in a human breath sample to nitric oxide (NO), the method comprising:
selecting a material catalytic for conversion of ammonia to nitric oxide;
forming a tube from the material, the tube having an inlet, an outlet, and a wall defining an internal surface of the tube and an external surface of the tube, the wall comprising the material along a thickness from the internal surface of the tube to the external surface of the tube; and
configuring a heater such that in use the heater heats the tube.

18. The method according to claim 17 wherein the material catalytic for conversion of ammonia to nitric oxide comprises stainless steel.

19. The method according to claim 17 comprising shaping the tube to have a ratio between a shortest distance within the tube from the inlet to the outlet and a shortest distance from the inlet to the outlet, the ratio being greater than or equal to 2:1.

20. The method according to claim 19, wherein the shaping of the tube comprises shaping the tube to have a substantially helical shape.

21. A method of measuring the concentration of ammonia (NH₃) in a human breath sample with a device for converting NH₃ in a human breath sample to nitric oxide (NO),
the method comprising:
heating a tube with a heater to a temperature equal to or greater than 600° C., the tube comprising an inlet, an outlet, and a wall comprising an internal surface of the tube and an external surface of the tube, the wall comprising substantially the same material along a thickness from the internal surface of the tube to the external surface of the tube, the material catalytic for conversion of ammonia to nitric oxide;
supplying the human breath sample to the inlet of the tube such that the human breath sample passes along the tube and through the outlet to a nitric oxide sensor in fluid communication with the outlet, wherein at least 90% of the ammonia present in the human breath sample is converted to nitric oxide before contacting the nitric oxide sensor; and
detecting the amount of nitric oxide present in the heated human breath sample with the nitric oxide sensor to provide a measurement of the concentration of ammonia in the human breath sample.

22. The method according to claim 21, wherein heating the tube comprises supplying power to the heater from a power supply, and the method comprises disconnecting the power supply from the heater before supplying the human breath sample to the tube.

23. The method according to claim 21 further comprising providing an indication of the rate at which the human breath sample is being supplied to the inlet of the tube.

24. The method according to any of claims 21, the method further comprising comparing the measurement of the concentration of ammonia in the human breath sample with an expected value.
